(19) United States
(12) Patent Application Publication
(10) Pub. No.: US 2010/0009389 A1
(43) Pub. Date: Jan. 14, 2010

(54) Method of Making and Using Versatile Positive Controls and Antibody Detection Assays

(76) Inventors: Masood Unnabi Khan, Potomac, MD (US); Rahat Jahan Khan, Potomac, MD (US)

Correspondence Address: PCT LAW GROUP, PLLC, 818 CONNECTICUT AVENUE, NW, SUITE 700, WASHINGTON, DC, 20006, US

(21) Appl. No.: 12/502,814

(22) Filed: Jul. 14, 2009

Related U.S. Application Data
(60) Provisional application No. 61/080,326, filed on Jul. 14, 2008.

(51) Int. Cl. G01N 33/573; G01N 33/536; G01N 33/543

(52) U.S. Cl. 435/7.9; 436/536; 436/518

(57) ABSTRACT

The present invention is in the technical field of ligand-binding assays for detection of antigen-specific antibodies. Specifically a method for preparation of a versatile positive control, components of which can be mixed and matched in numerous combinations to render positive controls for antigen specific antibodies of a wide variety of Immunoglobulin classes (e.g., IgG, IgA, IgM, IgE etc.) in both human and animals is described. Also disclosed are assay methods and kits in which the positive control components are used for detection of antigen-specific antibodies in biological and non-biological matrices.

### CROSS-REFERENCE TO RELATED APPLICATIONS

This application is a non-provisional of U.S. Appl. No. 61/080,326, filed Jul. 14, 2008. The content of the aforesaid application is relied upon and hereby incorporated by reference in its entirety.

### BACKGROUND OF THE INVENTION

The present invention is in the technical field of ligand-binding assays. More particularly, the present invention relates to: (a) methods of making versatile controls and calibrators that can be used in ligand binding assays, such as immunoassays; (b) to assay methods, in which said calibrators and controls are used, for detection of antigen-specific antibodies in biological matrices; and (c) to kits for determining the presence of an antibody in a test sample.

Ligand-binding assays, such as Enzyme-linked Immunosorbent Assay (ELISA), more specifically Sandwich ELISA methods are widely used for detection of antibodies against specific antigens associated with the infectious agents (e.g., bacteria, viruses), toxins, allergens, drugs and a variety of other large molecule biochemical entities that elicit the induction of antibody production in human or animal body. Both the qualitative and quantitative assays for detection of antibodies require both negative and positive controls. For the quantitative assays, it may also be necessary to have suitable calibrators (i.e., samples containing a known amount of antibody of interest) available in sufficient quantities. The positive control is used to monitor the integrity of the reagents and also to establish the assay performance characteristics. Typically negative controls are prepared from a normal subject that is screened to not to have the antibodies of interest. Positive controls are prepared by obtaining serum from subjects that are screened to have sufficiently high amount of antibodies of interest present in their sera. Positive calibrators are prepared, subsequently, by spiking the negative controls with appropriate amounts of antibody positive serum. However, this approach is not only labor intensive and costly but also has several inherent serious problems including: (a) potential safety risk of contamination of serum by pathogen; (b) difficulty in recruiting the blood donors with antibodies of interest; and (c) scarcity of subjects with antibodies of less abundant classes such as Immunoglobulin M (IgM) or Immunoglobulin F (IgE).

More recently, the need for detection of anti-drug antibodies (ADA) has emerged as an area of great interest to the pharmaceutical and biotechnology industries. Over the last 20 years, a new class of drugs, known as ‘biologics’ or ‘biotech drugs’, has emerged. These drugs are mostly based on the ‘recombinant proteins’ and ‘humanized monoclonal antibodies.’ Thus, the biotech drugs are large molecules, and therefore, they have intrinsic potential to produce ‘anti-drug-antibodies’ (ADA) in patients. These antibodies, in many cases, may be harmless (having no effect on the drug molecule) but some ADA may form complexes with the drug and in turn alter the pharmacokinetics of the drug. Some of them may even neutralize the bioactivity of the drug molecule making the drug ineffective. In cases where the biotech drugs are closely related to the natural molecules that are critical for normal functioning of the body, interactions of ADA with such molecule may induce serious adverse effects, creating a safety risk. In view of this, regulatory agencies require that testing of these antibodies be an integral part of pre-clinical (in animal) and clinical (in human) trials of biotech drugs. At this stage of development of the new drug, subjects are exposed to the drug for the first time. Hence, a genuine ‘positive control’, namely, a ‘human anti-drug antibody of class of interest’ or a ‘test animal ant-drug antibody of class of interest’ may not be available at all.

As stated earlier, antibody detection assays require both negative and positive controls. Unavailability of the genuine positive control poses a serious problem in designing an anti-drug antibody assay. However, in order to monitor the performance of ADA assay, especially in order to assure the coating of wells in the micro-titer plate with a correct type of drug molecule, an indirect positive control consisting of the non-human ‘anti-drug antibody’ (monoclonal or polyclonal) is used. This necessitates the use of two different detection reagents (FIGS. 1A & 1B), such as ‘labeled-antibody against non human Immunoglobulin of class of interest’ (for detection of non-human ADA in control sample) and a ‘labeled-antibody against human Immunoglobulin of class of interest’ (for detection of human ADA in test sample). This requires additional efforts in optimization and validation of using appropriate dilutions of two detection antibody reagents. Moreover, it does not provide a control for the appropriateness of the proper functioning of the ‘labeled-antibody against human Immunoglobulin of class of interest.’ If there is a positive signal in the wells for control sample, it would confirm that the plate has a correct antigen (drug) coating. However, on the same plate if there is no positive signal in the wells for the patient samples, it would not be evident if it is due to the absence of ADA in the patient sample or it is due to the failure of the detection reagent (i.e., ‘labeled-antibody against human Immunoglobulin of class of interest’) used.

In view of the above, it would be advantageous, and highly desirable to have ample amounts of positive controls available for antibody detection assays for a wide variety of entities, including, pathogens, allergens and large molecule biotech drugs. Such controls will be extensively utilized in the diagnostic industry as well as in drug development operations.

### DESCRIPTION OF THE RELATED ART

The information provided below is not admitted to be prior art to the present invention, but is provided solely to assist the understanding of the reader.

A number of previous descriptions of making artificial positive controls and calibrators are in existence.

Wong et al. (U.S. Pat. No. 5,008,183) describes the preparation and use of artificial positive control for IgM assays. The reagent consists of a covalently bound conjugate of a non-human antigen-specific antibody to a non-specific human IgM molecule. Thus in an IgM assay the specific antibody component will bind to the antigen immobilized on the solid surface while the human IgM component will be available to bind with the labeled anti-human IgM antibody used as detection reagent that will also bind to the antigen specific human IgM captured by the immobilized antigen from the patient sample, in the Microtiter plate wells designated for control and patient samples, respectively (FIG. 2). However, the disadvantages of this procedure include: elaborate purification of the conjugate, potential of formation of cross-linking between multiple molecules, reduction in binding of specific end of conjugate to the antigen confer by the steric hindrance resulting from the conjugation of multiple bulky molecules like IgM. Moreover, by design the covalent linking of ‘antigen specific antibody’ to a specific type of human immunoglobulin lock down the ‘antigen specific antibody’ to a specific type of positive control and will require separate chemical conjugation and purifications for preparation of each type of positive control that could be labor intensive and a less cost effective proposition.

Brust et al. (U.S. Pat. Nos. 5,491,218 and 5,895,811) describe an improvement over the invention of Wong et al. Their artificial positive control is a bi-functional conjugate consisting of two components—A and B. Where component A is an antigen (or pathogen) specific non-human antibody covalently linked to component B, another non-human antibody against immunoglobulin of same Ig-class as the analyte that is being detected. The bi-functional conjugate is spiked into normal human serum that ‘does not contain’ analyte specific antibodies of desired Ig-class. The bi-functional conjugate per se may not act as a positive control unless it is added to the normal human serum from which component B binds and capture the immunoglobulin of class that is intended to be detected (FIG. 3). For example, an artificial positive control for an Anti GM-CSF IgM assay, the component A could be a rabbit-anti GM-CSF antibody and component B could be a mouse-anti-human IgM antibody. As it is spiked into normal human serum (devoid of specific anti GM-CSF IgM), and used as ‘positive control’, the component A will bind to the immobilized GM-CSF, and the component B that has already captured non specific human IgM from normal human serum, will be ready to be detected by the labeled anti-human IgM antibody used for detection of human anti-GM-CSF IgM in the patient samples. The additional advantage of this invention over that described by Wong et al. is that the conjugate may be less bulky, for instance, in positive control for IgM, the component B is an Immunoglobulin (IgG) or a F(ab′) fragment which is less bulky and can capture human IgM in its native form from normal human serum. However, this conjugate remains non-versatile and other disadvantages remain essentially same as described above. Moreover, an additional disadvantage of this approach includes: if the analyte of interest is Ig of a class that is less abundant, such as IgE, it could make the positive control for IgE ineffective because of insufficient amounts of IgE captured by the conjugate.

Hackett Jr. et al. (U.S. Pat. No. 6,015,662) describes a genetically engineered chimeric antibody that binds specifically to a predetermined antigen, and also contains antibody constant region epitopes, and is uniform in specificity and affinity. For example for a positive control in a human anti-GM-CSF IgM assay, a recombinant mouse-human chimeric antibody can be constructed that will be captured by the GM-CSF immobilized on the microtiter plate and will also be detected by the labeled anti-human IgM used for detection of human anti-GM-CSF IgM antibody in the patient sample. Similarly this approach has huge disadvantages including; the process of designing and producing such antibodies is complex, elaborate, and costly. It requires specialized skills and equipment. It may not be practical and cost effective to produce positive controls in small quantities and in a short period of time.

### BRIEF SUMMARY OF THE INVENTION

The present invention provides methods for the preparation of a versatile positive control, components of which can be mixed and matched in numerous combinations to render positive controls for antigen specific antibodies of a wide variety of Immunoglobulin classes (e.g., IgG, IgM, IgE, etc.) in both human and animals. The present invention also provides an antibody detection assay and kits that utilize said positive controls.

An aspect of the present invention provides a positive control/calibrator for use in an antibody detection assay that does not have the major disadvantages associated with conjugates of the prior art.

An aspect of the present invention provides a positive control that is versatile, does not require specialized expensive equipments or specialized skills, and can be easily prepared in small or large quantities.

An aspect of the present invention provides an ‘antibody detection assay’ which utilizes the positive control/calibrator of said invention.

An aspect of the present invention provides a positive control composition that can be used for creating assay calibrators. Therefore, the term ‘positive control’ will also mean a calibrator.

The present invention encompasses a non-covalently-linked positive control reagent for an immunological assay comprising: (i) an antigen-specific antibody, wherein said antigen-specific antibody comprises a first bound affinity reagent; (ii) a linker immunoglobulin, wherein said linker immunoglobulin comprises a second bound affinity reagent that binds to said first bound affinity reagent of part (i); and (iii) a detector antibody that binds to said linker immunoglobulin of part (ii), wherein said detector antibody comprises a signal generator and wherein said antibodies of parts (i) and (iii) are from a different species than the immunoglobulin of part (ii).

The present invention further encompasses a method of performing a positive control for an immunological assay comprising: (i) providing a solid support having an antigen bound to a surface thereof, (ii) providing an antibody that is specific for said antigen of part (i), wherein said antibody comprises a first bound affinity reagent; (iii) binding said antibody of part (ii) to said antigen of part (i); (iv) providing a linker immunoglobulin, wherein said linker immunoglobulin comprises a second bound affinity reagent that binds to said first bound affinity reagent of part (ii); (v) binding said first and second affinity reagents; (vi) providing a detector antibody that binds to said linker immunoglobulin of part (iv), wherein said detector antibody comprises a signal generator; (vii) binding said detector antibody of part (vi) to said linker immunoglobulin of part (iv); and (viii) generating a signal with said signal generator wherein said antibodies of parts (ii) and (vi) are from a different species than the immunoglobulin of part (iv).

The present invention also encompasses a kit for determining the presence of an antigen-specific antibody in a test sample comprising: (i) an antigen specific for the antibody; (ii) an antigen-specific antibody, wherein said antigen-specific antibody comprises a first bound affinity reagent; (iii) a linker immunoglobulin, wherein said linker immunoglobulin comprises a second bound affinity reagent that binds to said first bound affinity reagent of part (ii); and (iv) a detector antibody that binds to said linker immunoglobulin of part (iii), wherein said detector antibody comprises a signal generator wherein said antibodies of parts (ii) and (iv) are from a different species than the immunoglobulin of part (iii). The antigen can be provided in the kit either in unbound form or coated on a solid surface which can be included in the kit.

An aspect of the present invention provides a positive control composition comprising first and second immunoglobulin molecules of two components (Component A and Component B), wherein each component is labeled with a complementary bio-affinity molecule. An aspect of the present invention provides where the complementary bio-affinity molecule are brought into contact, they form a strong, non-covalent complex.

An aspect of the present invention provides a first component (Component A) capable of specifically binding to a pre-determined antigen, for example an antibody against ‘antigen of interest.’ An aspect of the present invention provides the first component is labeled with a first bio-affinity molecule, such as biotin or a derivative of biotin.

An aspect of the present invention provides a second component (Component B or Linker Immunoglobulin). An aspect of the present invention provides the second component is a ‘non-specific immunoglobulin’ of the same Ig-class as the ‘analyte of interest.’ An aspect of the present invention provides the second component is labeled with a second bio-affinity molecule, such as Avidin, Streptoavidin or a derivative thereof.

An aspect of the present invention provides a positive control composition and an assay method which can be used to any sandwich type antibody detection ligand-binding assay.

Still other aspects and advantages of the present invention will become readily apparent by those skilled in the art from the following detailed description, wherein it is shown and described preferred aspects of the invention, simply by way of illustration of the best mode contemplated of carrying out the invention. As will be realized the invention is capable of other and different aspects, and its several details are capable of modifications in various obvious respects, without departing from the invention. Accordingly, the description is to be regarded as illustrative in nature and not as restrictive.

### BRIEF DESCRIPTION OF THE FIGURES

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following seven figures:

FIG. 1 depicts the current use of artificial positive control in an Anti-drug Antibody (ADA) immunoassay where two different detection reagents are required. Sera from an animal (e.g., Rabbit) hyper immunized with Drug (or Pathogen) is used as Positive Control. Two different Detection Reagents are required, e.g., for detection of Human ADA (e.g., drug specific IgG) in Test Samples, a Labeled Anti Human IgG (e.g., Rabbit Anti Human IgG-Enzyme) is required as detection reagent. For Rabbit ADA (e.g., drug specific IgG) in Positive Control a Labeled Anti Rabbit IgG (e.g., Goat Anti-Rabbit IgG-Enzyme) is required as detection reagent.

More specifically, FIG. 1A depicts Human ADA in Test Sample, such as human ADA of type IgG, captured by immobilized drug detected by Enzyme labeled Anti-human IgG, such as Rabbit Anti-human IgG-Enzyme. And more specifically, FIG. 1B depicts a non-human Positive Control, where the non-human ADA (such as Rabbit ADA IgG) captured by immobilized drug is detected by Enzyme labeled Anti-non-human IgG (such as Goat Anti-Rabbit IgG-Enzyme).

FIG. 2 depicts a conventional artificial positive control which is bi-functional conjugate. One component of the conjugate is a drug (or pathogen) specific antibody and the second component is a non specific immunoglobulin of the immunoglobulin class to be detected in the assay (Analyte). Both components are chemically linked to form a bulky conjugate.

FIG. 3 depicts a conventional artificial positive control which is bi-functional conjugate. One component of the conjugate is a drug (or pathogen) specific antibody and the second component is an antibody directed against Analyte i.e., immunoglobulin of the immunoglobulin class to be detected in the assay. Both components are chemically linked to form a functional bulky conjugate that captures the non-specific analyte from the serum in order to act as a positive control for analyte detection.

FIG. 4A depicts an aspect of non-human anti drug antibody 400A that is the first component (Component A) of the claimed invention. Anti-drug antibody comprises at least one antigen binding site 401 having a high affinity for antigen or drug. Anti-drug antibody is covalently modified by at least one first affinity reagent 402.

FIG. 4B depicts an aspect of second component 400B of the claimed invention. Second component (Component B) is a non-specific analyte immunoglobulin comprises at least one antigenic determinant 403 for which detector antibody has a high affinity. Second component 400B is covalently modified by at least one second affinity reagent 404.

FIG. 4C depicts an aspect of a detector antibody 400C. Detector antibody 400C has at least one binding site 406 having a high affinity for antigenic determinant 403. Detector antibody 400C is covalently modified by at least one signal generator 405.

FIG. 5 depicts first component, that is non-human anti drug antibody 400A non-covalently bound to antigen (drug) 501 immobilized on the solid surface 502.

FIG. 6 depicts non-human anti drug antibody (first component) 400A immobilized to surface 502 by binding to antigen 501 and non-covalently bound to second component 400B via the interaction of first affinity reagent 402 and second affinity reagent 404.

FIG. 7 depicts detector antibody 400C non-covalently bound to the complex of FIG. 6. Antibody 400C binds via the interaction of binding site 406 with antigenic determinant 403. It is to be noted, however, that the appended drawings illustrate only typical aspects of this invention and are therefore not to be considered limiting of its scope, for the invention may admit to other equally effective aspects.

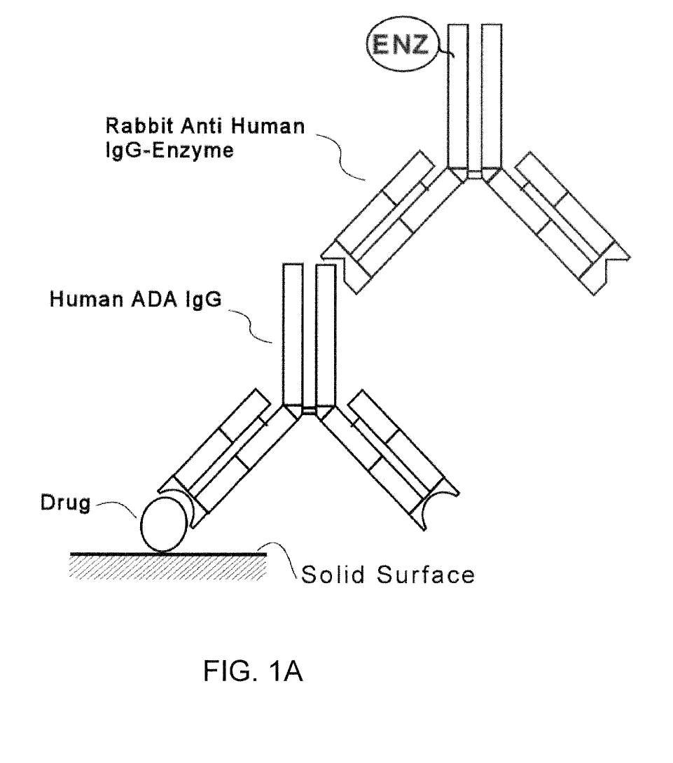

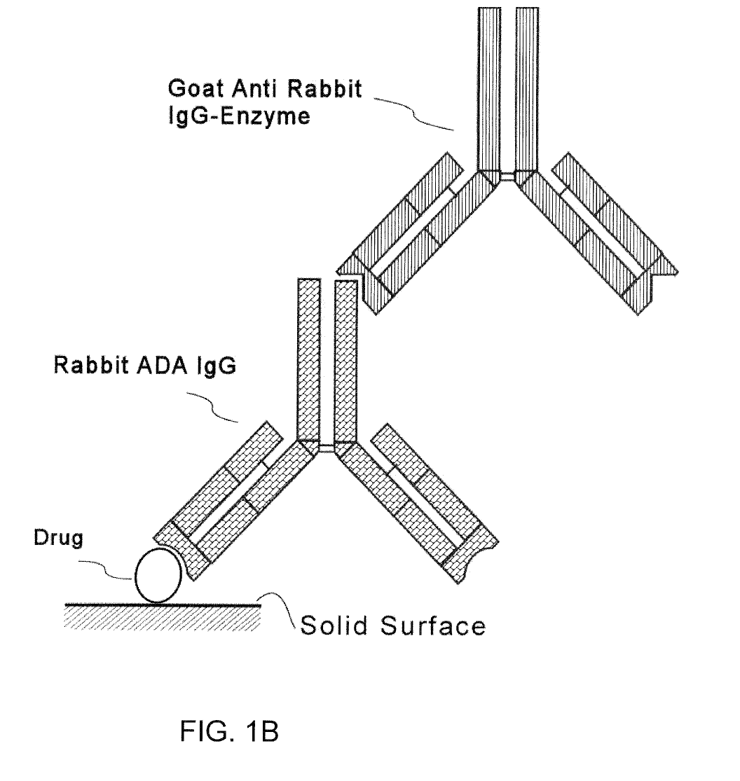

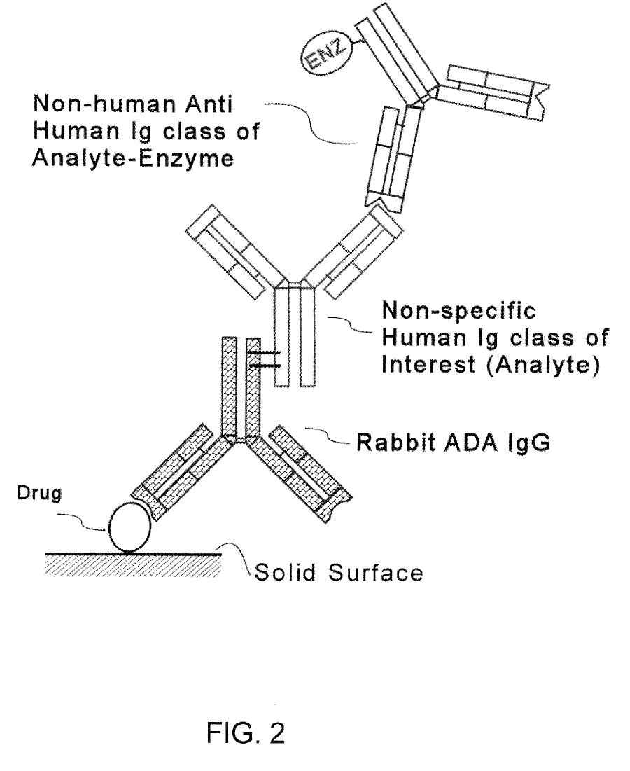

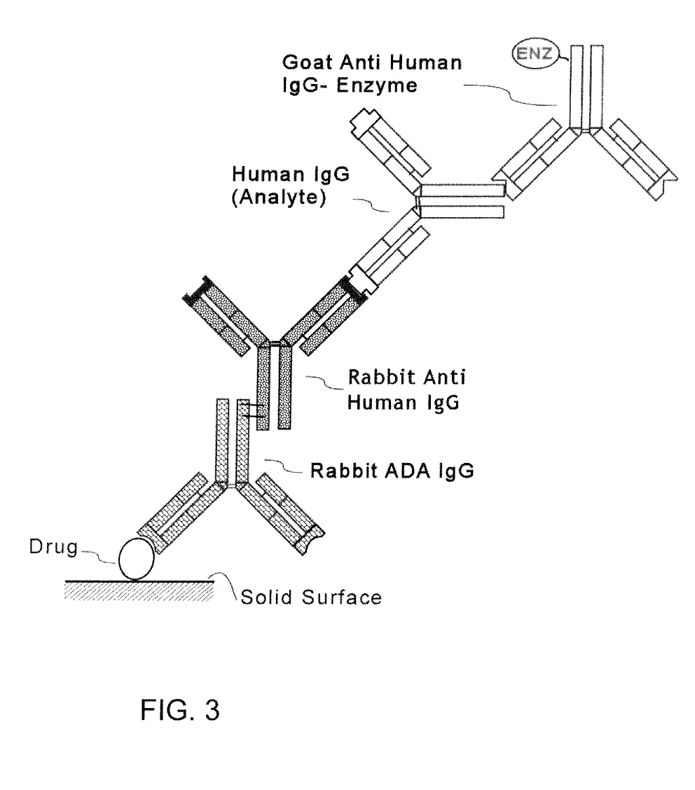

### DETAILED DESCRIPTION OF THE INVENTION

Reference is made to the figures to illustrate selected aspects and modes of carrying out the invention. It is to be understood that the invention is not hereby limited to those aspects depicted in the figures.

As used herein the term antibody or ‘antibodies’ include both polyclonal and monoclonal antibodies, whole immunoglobulin molecules and antigen binding fragment(s) of the molecule. Such fragments can be produced by the methods known in the art. Antigen binding fragments comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); and single-chain antibody molecules.

As used herein, the term ‘linker immunoglobulin’ in accordance with the invention includes both whole immunoglobulin molecules and fragments thereof. As will be appreciated by one skilled in the relevant art(s), in accordance with the invention, such fragments of the linker immunoglobulin may or may not include portions of the molecule that bind to antigens. Further, as will be appreciated by one skilled in the relevant art(s), in accordance with the invention, it is not necessary for the linker immunoglobulin to have a characterized specificity for a known antigen.

The antibodies or linker immunoglubulins of the present invention can be derived from any source. Examples of antibody or immunoglobulin sources include, but are not limited to, rodent (e.g., mouse, hamster, rat), chicken, rabbit, canine, feline, bovine, equine, porcine, chimpanzee, goat and human.

As used herein the term ‘analyte of interest’ means the antigen-specific antibody of a specific Immunoglobulin (Ig) class, for example, for ‘human anti-GM-CSF IgM.’, the ‘analyte of interest’ is the ‘human antibody of IgM-class of immunoglobulin’ that is specific to GM-CSF, whereas, ‘GM-CSF’ is the ‘antigen of interest’In the context of the present invention, the term ‘antigen’ means any agent that may elicit the induction of antibody production in a human or an animal body. By way of non-limiting example, the tern antigen includes infectious agents (e.g., bacteria, viruses, etc.), toxins, allergens, and large molecule biochemical entities such as peptides, proteins, carbohydrates and nucleic acids. The term antigen may also include drugs and other complexed small molecules that induce antibody production.

The present invention encompasses a non-covalently-linked positive control reagent for an immunological assay comprising: (i) an antigen-specific antibody, wherein said antigen-specific antibody comprises a first bound affinity reagent; (ii) a linker immunoglobulin, wherein said linker immunoglobulin comprises a second bound affinity reagent that binds to said first bound affinity reagent of part (i); and (iii) a detector antibody that binds to said linker immunoglobulin of part (ii), wherein said detector antibody comprises a signal generator and wherein said antibodies of parts (i) and (iii) are from a different species than the immunoglobulin of part (ii).

In some aspects, the antigen specific antibody and the detector antibody are from the same species. In other aspects, the antigen-specific antibody and detector antibody are from different species.

The present invention further encompasses a method of performing a positive control for an immunological assay comprising: (i) providing a solid support having an antigen adsorbed to a surface thereof, (ii) providing an antibody that is specific for said antigen of part (i), wherein said antibody comprises a First bound affinity reagent; (iii) binding said antibody of part (ii) to said antigen of part (i); (iv) providing a linker immunoglobulin, wherein said linker immunoglobulin comprises a second bound affinity reagent that binds to said first bound affinity reagent of part (ii); (v) binding said first and second affinity reagents; (vi) providing a detector antibody that binds to said linker immunoglobulin of part (iv), wherein said detector antibody comprises a signal generator; (vii) binding said detector antibody of part (vi) to said linker immunoglobulin of part (iv); and (viii) generating a signal with said signal generator wherein said antibodies of parts (ii) and (vi) are from a different species than the immunoglobulin of part (iv).

The present invention also encompasses a kit for determining the presence of an antibody in a test sample comprising: (i) an antigen specific for the antibody; (ii) an antigen-specific antibody, wherein said antigen-specific antibody comprises a first bound affinity reagent; (iii) a linker immunoglobulin, wherein said linker immunoglobulin comprises a second bound affinity reagent that binds to said first bound affinity reagent of part (ii); and (iv) a detector antibody that binds to said linker immunoglobulin of part (iii), wherein said detector antibody comprises a signal generator and wherein said antibodies of parts (ii) and (iv) are from a different species than the linker immunoglobulin of part (iii). In some aspects, the kit further provides a solid surface for coating the antigen. In some aspects, the kit is provided with antigen already bound to the solid surface, such as a microtiter plate. In other aspects, the kit is provided with the antigen in unbound form. In some aspects, the experimenter coats the antigen on the solid surface.

In some aspects, the antigen is a recombinant protein or monoclonal antibody. In some aspects, the monoclonal antibody is humanized. In some aspects, the antigen is a monoclonal antibody that recognizes VEGF (e.g., bevacizumab), TNF-α (e.g., adalimnumab, infliximab), CD20 (e.g., rituximab), HER2/neu receptor (e.g., trastuzumab), T-cell receptor (e.g., muromonab), IgE (e.g., omalizumab), glycoprotein IIb/IIIa receptor (e.g., abciximab), antigenic site of the F protein of the Respiratory Syncytial Virus (RSV) (e.g., palivizumab). In some aspects, the antigen is a recombinant protein. In some aspects, the antigen is insulin glargine, etanercept or recombinant human growth hormone (e.g., PROTROPIN). In some aspects, the antigen is selected from the group consisting of AMEVIVE (alefacept), ZEVALIN (ibritumomab tiuxetan), ENBREL (etanercept), REOPRO (abeiximab), SIMULECT (basiliximab), SYNAGIS (palivizumab), ZENAPAX (daclizumab), CAMPATH (alemtuzumab), MYLOTARG (gemtuzumab ozogamicin), AVONEX (Interferon β-1a) and TYSABRI (natalizumab).

In accordance with the present invention, the antibodies or linker immunoglobulins of the present invention include IgA, IgD, IgE, IgG and IgM molecules or fragments thereof.

The antibody detection method of this invention is based on the Sandwich ligand-binding assay format where analyte of interest is sandwiched between immobilized antigen and detection reagent. In this format the specific antigen of interest (e.g., drug or pathogen) is immobilized on a solid-surface, such as, microtiter plate containing wells pre-designated for the control, calibrators or biological samples. The ‘biological sample’ which is suspected to have the ‘analyte of interest’ or the ‘Component A’ of said ‘positive control’, are added to pre-designated wells on the antigen-coated microtiter plate for a period of time to allow the capture of the ‘analyte of interest’ and the ‘Component A’ by the immobilized antigen. If needed a calibration curve can also be included in the assay by using varying concentrations of ‘Component A.’ Followed by washing the plate, ‘Component B’ of said ‘positive control’ is added to all or to only positive control designated wells and allowed to incubate for a period of time to allow the binding and capture of the ‘Component B’ by immobilized ‘Component A’ in the positive control wells. Followed by washing of the plate, the ‘detection reagent’ comprises of a label moiety that can be directly or indirectly detectable, linked to a detection material that can bind to the ‘analyte of interest’ is added to all wells. After a suitable period of incubation time, the plate is washed to remove un-bound detection reagent. Depending upon the nature of the label moiety the signal is measured using an appropriate procedure that should be known to one skilled in the relevant art(s). Typically, the intensity of the signal will be proportional to the amount of ‘analyte of interest’ in the biological sample, or amount of ‘Component A-Component B’ complex in the positive control.

The present invention provides a positive control for an immunoassay. An aspect of the present invention provides a rigid or semi-rigid substrate 503. In some aspects, substrate 503 permits the transmission of at least one wavelength of light. In another aspect, substrate 503 permits the substantial transmission of a broad spectrum of light. In some aspects, substrate 503 is glass or plastic.

In some aspects, an antigen 501 is bound to substrate 503. Antigen 501 may be bound by non-covalent, covalent, or a mix of covalent and non-covalent bonds.

The invention provides an antigen-specific antibody or ‘Component A’ 400A. Component A 400A has at least one high-affinity binding site 401 directed against antigen 501. Component A 400A has at least one first affinity reagent 402 attached. Affinity reagent 402 can be covalently or non-covalently attached to Component A. Reagent 402 is attached such that it does not interfere with binding of site 401 to antigen 501. Preferably, reagent 402 is attached to a region of antigen-specific antibody 400A remote from site 401.

In some aspects, antigen-specific antibody 400A may be derived from cloning or nonhuman species. In some aspects, antigen specific antibody 400A is derived from a non-human species.

In some aspects, antigen specific antibody 400A may be a polyclonal or monoclonal antibody or a fragment thereof so long as the fragment retains the capacity to specifically bind to antigen 501. In some aspects, antigen specific antibody 400A is a monoclonal antibody.

Antigen specific antibody 400A may be raised or produced according to any of the various methods that are well known to those skilled in the art.

Affinity reagent 402 may be either biotin or Avidin. In the context of the present invention, the term ‘biotin’ includes any natural and/or synthetic derivatives that manifest the property of strong binding to Avidin. In the context of the present invention, the term ‘Avidin’ includes any natural and/or synthetic derivatives (such as Streptavidin or NeutrAvidin) that manifest the property of strong binding to biotin. In some aspects, reagent 402 is biotin.

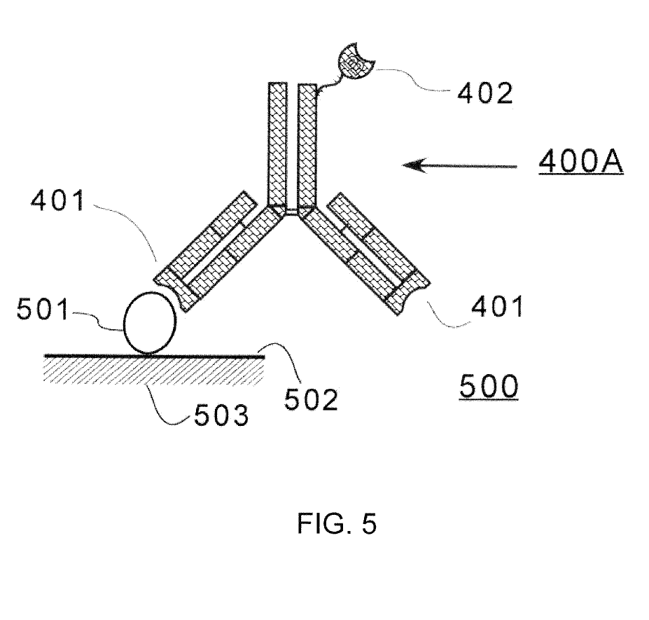

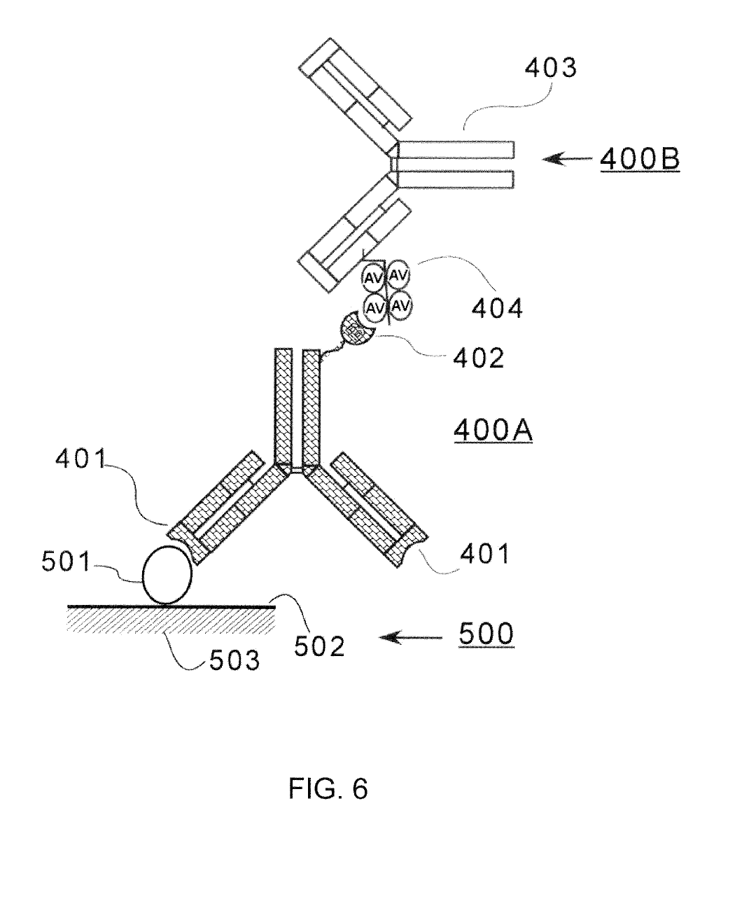

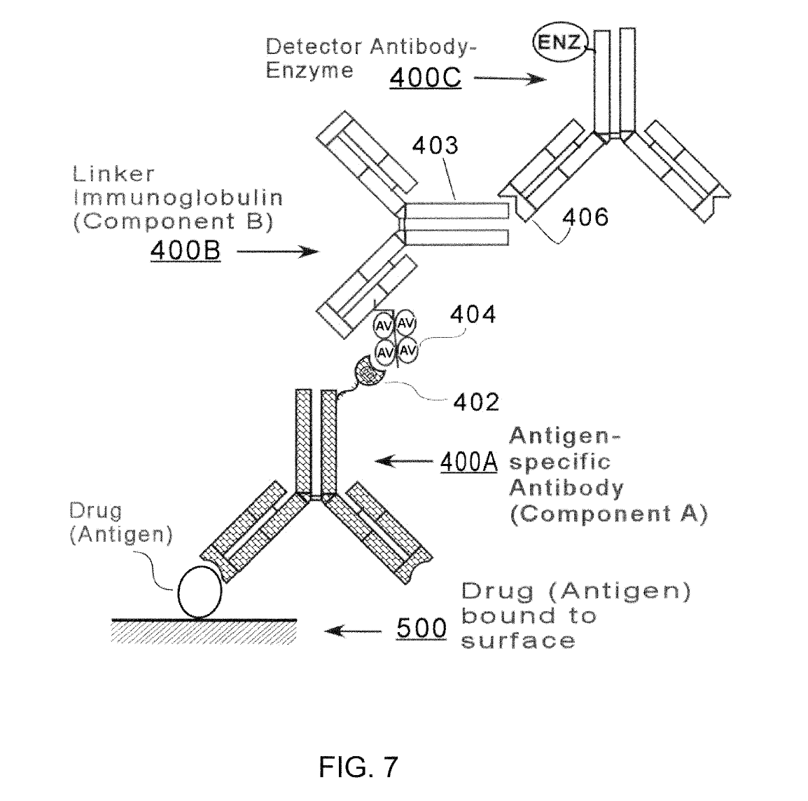

FIG. 5 depicts antigen 501, bound to a surface 502 of substrate 503. In some aspects of the invention, antigen 501, substrate 503, and antibody 400A are separately provided. In such an aspect, a user binds, adsorbs, or otherwise affixes antigen 501 to surface 502 and then binds antibody 400A to antigen 501. In another aspect, substrate 503 is provided with antigen 501 bound thereto and antibody 400A is separately provided. In such an aspect, a user binds antibody 400A to antigen 501. In a third aspect, antigen 501, substrate 503, and antibody 400A are provided substantially as shown in FIG. 5 with antigen 501 bound to surface 502 and antibody 400A bound to antigen 501.

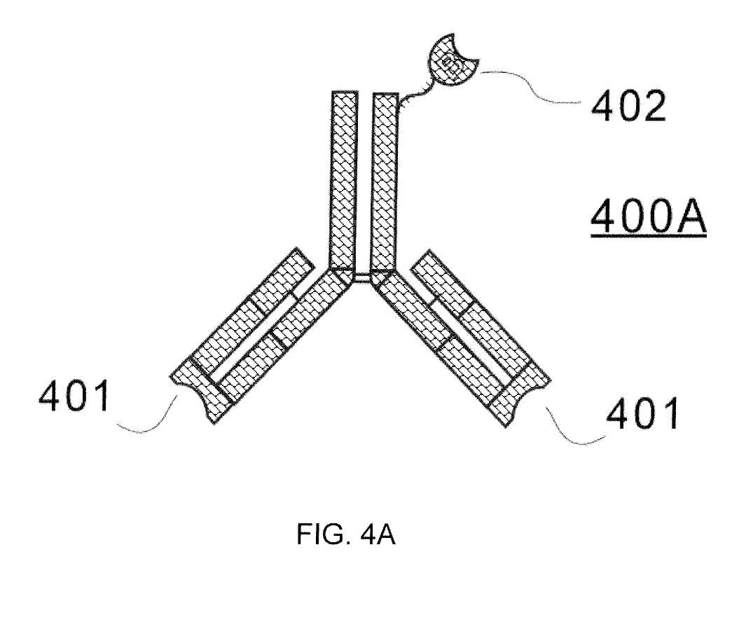

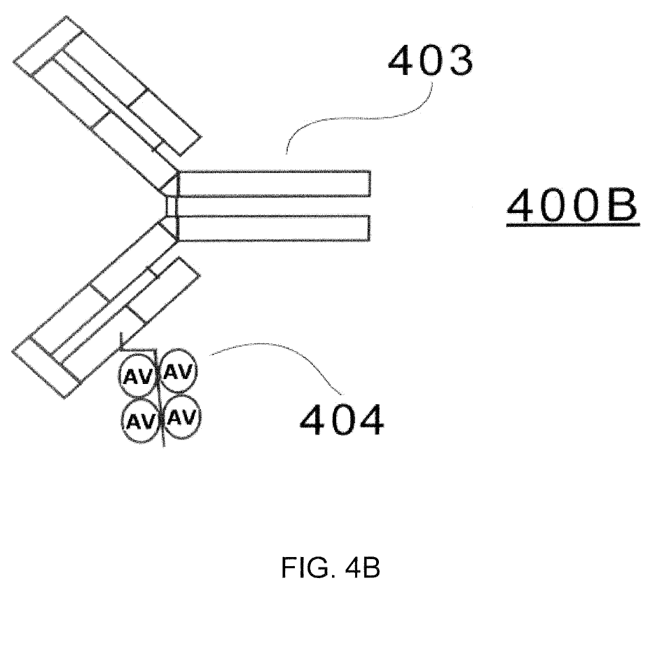

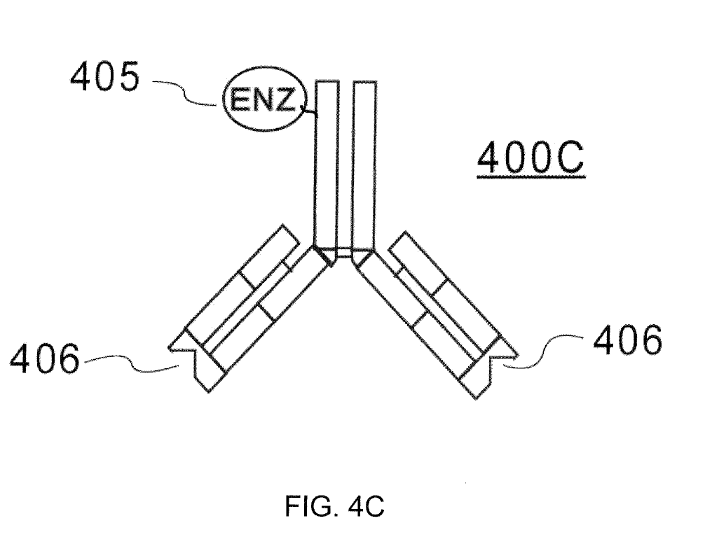

As shown in FIG. 4B, the invention provides a ‘Component B’ 400B. Component B 400B has a second affinity reagent 404 attached thereto. The second affinity reagent 404 can be covalently or non-covalently bound to ‘Component B.’ Second reagent 404 is complementary to, and forms a strong, non-covalent bond with first affinity reagent 402. In some aspects, the second affinity reagent 404 may be either Avidin or biotin whichever is complementary to the first affinity reagent 402. In some aspects, the first affinity reagent is biotin and the second affinity reagent is Avidin.

‘Component B’ 400B is derived from a species S1 different from the species S2 from which the antigen-specific antibody, that is, ‘Component A’ 400A is derived. In some aspects, ‘Component B’ 400B is of human origin and antigen-specific antibody 400A is derived from a non-human species. ‘Component B’ 400B comprises at least one antigenic determinant 403 which is not present on antigen-specific antibody 400A.

‘Component B’ 400B is a non-specific immunoglobulin with respect to each of antigen 501 and antigen-specific antibody 400A. ‘Component B’ 400B has at most a negligible binding affinity to either antigen 501 or antigen-specific antibody 400A. ‘Component B’ 400B may be of any immunoglobulin class. ‘Component B’ 400B is of the same immunoglobulin class as the ‘analyte of interest.’

‘Component B’ 400B may be of human or non-human origin provided that ‘Component B’ 400B and antigen-specific antibody 400A are not derived from the same species. ‘Component B’ 400B may be an intact immunoglobulin molecule or may be an Fe or other fragment provided that at least one antigenic determinant 403 is retained. ‘Component B’ 400B may be isolated and purified according to methods that are well known to those skilled in the art.

The conjugation or attachment of affinity labels to antigen-specific antibody 400A and/or ‘Component B’ 400B may be achieved using methods familiar to the one skilled in the relevant art(s).

As shown in FIG. 4C, the invention provides a detector antibody 400C. Detector antibody 400C is derived from an animal species S3 different from the species S1 from which ‘Component B’ 400B is derived. In some aspects, ‘Component B’ 400B is of human origin and detector antibody 400C is derived from a non-human species. The species S3, from which the detector antibody is derived, may be the same as, or different from, S2, the species from which the antigen-specific antibody is derived. Detector antibody 400C has at least one binding site 406 having a high affinity for antigenic determinant 403. Detector antibody 400C has, at most, a negligible binding affinity for antigen-specific antibody 400A. In some aspects, detector antibody 400C is modified by at least one signal generator 405. The signal generator 405 can be covalentlty or non-covalently attached.

A variety of signal generating enzymes and enzyme substrate pairs can be used. Most common enzymes include peroxidase, alkaline phosphatase, β-D-galactosidase, with corresponding substrates like H₂O₂/chromogen, 4-nitro-phenol, and 4-methylumbelliferone, respectively. Non enzymatic, radioactive signal generators, such as ¹²⁵I or ³H, can also be used. (See ‘Enzyme Immunoassays From Concept to Product Development’, by S. S. Deshpande, Chapman & Hill, New York, 1996.)

The present invention will be further illustrated by the following non-limiting examples.

### EXAMPLES

### Example 1

### Microtiter ELISA Preferred Aspect of the Invention

By way of non-limiting example, the present invention may be embodied as a microtiter plate-based ELISA. Microtiter plate-based ELISA assays are standard in the art. (See ‘Enzyme Immunoassays From Concept to Product Development’, by S. S. Deshpande, Chapman & Hill, New York, 1996.)

FIG. 5 shows antigen 501 bound to surface 502 of substrate 503. Substrate 503 may be a microtiter plate, but other substrates are within the invention. Antigen 501 may be a drug, pathogen or other molecule. Antigen 501 may be a peptide, a protein or any other immunogenic molecule. Antigen 501 may be bound to surface 502 using standard procedures known to one skilled in the relevant art(s). Unoccupied binding sites on surface 502 may be blocked using a material that is not bound by antigen-specific antibody 400A using standard procedures known to one skilled in the relevant art(s).

Some wells of the microtiter plate (substrate 503) may be assigned as test wells. A test sample may be applied to the test wells. The test sample may be unfractionated or partially-fractionated serum or may be a purified antibody or fraction thereof. Preferably, the test sample contains an antibody, or fragment thereof, directed against antigen 501. In some aspects, the antibody directed against antigen 501 is derived from a human. The test sample may be incubated for a defined time period, such as one hour at room temperature using procedures standard to the art.

Some wells of the microtiter plate (substrate 503) may be assigned as positive control wells. A positive control sample may be applied to the positive control wells.

A positive control sample containing antigen-specific antibody (i.e., ‘Component A’) 400A may be added to the positive control wells. In some aspects, the positive control sample is incubated similarly to the test sample.

Some wells of the microtiter plate (substrate 503) may be assigned as negative control wells. A negative control sample may be applied to the negative control wells and incubated similarly to the test and positive control samples. A negative control is a serum or buffer sample known to be devoid of antibody material that cross-reacts with antigen 501.

Following incubation, the wells may be washed to remove un-bound material.

‘Component B’ 400B is added to the positive control wells where it will bind to the ‘Component A’ 400A captured by antigen 501, immobilized on wells. ‘Component B’ 400B may also be added to the test and/or negative control wells where it will not bind to wells. ‘Component B’ 400B may be diluted with serum or buffer, provided that the diluent does not contain an antibody material that binds to antigen 501. The ‘Component B’ may be incubated at room temperature for one hour or otherwise as is known to one skilled in the relevant art(s).

Following incubation, the wells may be washed to remove un-bound material.

Detector antibody 400C is added to each well and incubated as is known in the art. By way of non-limiting example detector antibody 400C may be incubated for one hour at room temperature.

Following incubation, the wells may be washed to remove un-bound material.

A suitable enzyme substrate is added to each well and incubated under conditions of time and temperature that are readily apparent to one skilled in the relevant art(s). Following the incubation, the enzymatic reaction is stopped and the extent of enzymatic reaction is quantified by methods that are readily apparent to one skilled in the relevant art(s). For example, the product of an enzymatic reaction may be quantified spectrophotometrically at an appropriate wavelength using Beer's Law. (See, e.g., ‘Enzyme Immunoassays From Concept to Product Development’, by S. S. Deshpande, Chapman & Hill, New York, 1996.)

### INDUSTRIAL UTILITY

This invention has industrial applicability in providing a positive control reagent for an antigen-specific antibody ELISA, and methods of using same.

### INCORPORATION BY REFERENCE

Throughout this application, various references including publications, patents, and pre-grant patent application publications are referred to. Disclosures of these publications in their entireties are hereby incorporated by reference into this application to more fully describe the state of the art to which this invention pertains. It is specifically not admitted that any such reference constitutes prior art against the present application or against any claims thereof. All publications, patents, and pre-grant patent application publications cited in this specification are herein incorporated by reference, and for any and all purposes, as if each individual publication or patent application were specifically and individually indicated to be incorporated by reference. In the case of inconsistencies the present disclosure will prevail.

We claim:

1. A non-covalently-linked positive control reagent for an immunological assay, comprising:
(i) an antigen-specific antibody, wherein said antigen-specific antibody comprises a first bound affinity reagent;
(ii) a linker immunoglobulin, wherein said linker immunoglobulin comprises a second bound affinity reagent that binds to said first bound affinity reagent of part (i); and
(iii) a detector antibody that binds to said linker immunoglobulin of part (ii), wherein said detector antibody comprises a signal generator;
wherein said antibodies of parts (i) and (iii) are from a different species than the immunoglobulin of part (ii).

2. The positive control reagent of claim 1, wherein said first affinity reagent comprises biotin, and wherein said second affinity reagent comprises Avidin.

3. The positive control reagent of claim 1, wherein said first affinity reagent comprises Avidin, and wherein said second affinity reagent comprises biotin.

4. The positive control reagent of claim 1, wherein said signal generator comprises an enzyme.

5. The positive control reagent of claim 4, wherein said enzyme is selected from the group consisting of peroxidase, phosphatase and galactosidase.

6. The positive control reagent of claim 4, wherein said enzyme is a signal amplification enzyme.

7. The positive control reagent of claim 1, wherein said signal generator comprises a radioactive or non-radioactive label.

8. The positive control reagent of claim 1, wherein said antigen-specific antibody of part (i) is an immunoglobulin or fragment thereof selected from the group consisting of a monoclonal antibody and a polyclonal antibody.

9. The positive control reagent of claim 1, wherein said linker immunoglobulin of part (ii) is an immunoglobulin or fragment thereof selected from the group consisting of IgG, IgM, IgA, IgD, and IgE.

10. The positive control reagent of claim 1, wherein said detector antibody of part (iii) is an immunoglobulin or fragment thereof selected from the group consisting of a monoclonal antibody and a polyclonal antibody.

11. The positive control reagent of claim 1, wherein said antigen-specific and detector antibodies are non-human antibodies, and said linker immunoglobulin is human.

12. The positive control reagent of claim 11, wherein said first bound affinity reagent is covalently-bound to said antigen-specific antibody, and said second bound affinity reagent is covalently-bound to said linker immunoglobulin.

13. The positive control reagent of claim 1, wherein said antigen specific and detector antibodies are non-human antibodies.

14. A method of performing a positive control for an immunological assay, comprising:
(i) providing a solid support having an antigen adsorbed to a surface thereof;
(ii) providing an antibody that is specific for said antigen of part (i), wherein said antibody comprises a first bound affinity reagent;
(iii) binding said antibody of part (ii) to said antigen of part (i);
(iv) providing a linker immunoglobulin, wherein said linker immunoglobulin comprises a second bound affinity reagent that binds to said first bound affinity reagent of part (ii);
(v) binding said first and second affinity reagents;
(vi) providing a detector antibody that binds to said linker immunoglobulin of part (iv), wherein said detector antibody comprises a signal generator;
(vii) binding said detector antibody of part (vi) to said linker immunoglobulin of part (iv); and
(viii) generating a signal with said signal generator;
wherein said antibodies of parts (ii) and (vi) are from a different species than the immunoglobulin of part (iv).

15. The method of claim 14, wherein said antibody that is specific for said antigen and said detector antibody are non-human antibodies, and said linker immunoglobulin is human.

16. The method of claim 15, wherein said first bound affinity reagent is covalently-bound to said antigen-specific antibody, and said second bound affinity reagent is covalently-bound to said linker immunoglobulin.

17. A kit for determining the presence of an antibody in a test sample, comprising:
(i) an antigen specific for the antibody;
(ii) an antigen-specific antibody, wherein said antigen-specific antibody comprises a first bound affinity reagent,
(iii) a linker immunoglobulin, wherein said linker immunoglobulin comprises a second bound affinity reagent that binds to said first bound affinity reagent of part (ii); and
(iv) a detector antibody that binds to said linker immunoglobulin of part (iii), wherein said detector antibody comprises a signal generator;
wherein said antibodies of parts (ii) and (iv) are from a different species than the immunoglobulin of part (iii).

18. The kit of claim 17, wherein said antigen is a recombinant protein.

19. The kit of claim 17, wherein said first affinity reagent comprises Avidin, and wherein said second affinity reagent comprises biotin.

20. The kit of claim 17, wherein said antigen is a humanized monoclonal antibody.
